(19) United States
(12) Patent Application Publication
(10) Pub. No.: US 2005/0209526 A1
(43) Pub. Date: Sep. 22, 2005

(54) Animal monitoring device

(75) Inventors: Herbert A. Ingley III, Gainesville, FL (US); Carlos A. Risco, Gainesville, FL (US)

Correspondence Address: AKERMAN SENTERFITT, P.O. BOX 3188, WEST PALM BEACH, FL, 33402-3188, US

(73) Assignee: University of Florida, Gainesville, FL (US)

(21) Appl. No.: 11/010,988

(22) Filed: Dec. 13, 2004

Related U.S. Application Data
(60) Provisional application No. 60/528,730, filed on Dec. 11, 2003.

(51) Int. Cl. A61B 5/00; A61B 5/08

(52) U.S. Cl. 600/529; 600/549

(57) ABSTRACT

An animal monitoring device. In particular, the monitoring device is capable of not only identifying the animal and keeping track of its movement, but it may also be capable of monitoring ketone emissions and/or other vital health indicators of the animal. By tracking these measurements, it is possible to provide a unique indication of the health of the animal. Through data logging hardware, this device would be able log these clinical health indicators for a sufficient period of time in a non-invasive manner.

### CROSS REFERENCE TO RELATED APPLICATIONS

The present invention claims priority to U.S. Provisional Patent Application No. 60/528,730, filed Dec. 11, 2003.

### STATEMENT REGARDING FEDERALLY SPONSORED RESEARCH OR DEVELOPMENT

Not applicable.

### FIELD OF THE INVENTION

The present invention is directed to an article of manufacture useful as an animal monitoring device. In particular, the present invention is an animal monitoring device that may be attached to an animal.

### BACKGROUND OF THE INVENTION

A particular problem in caring for livestock, particularly livestock maintained in large herds in corrals, cattle pens and feed lots, is the detection of a sick animal at an early stage of illness. In the past, detection was generally accomplished by visual observation of the herd. In such a situation, the experience and expertise of the individual observing the herd was important as only well-trained persons could detect ailing animals unless the animal was in an advanced stage of illness. By this time, the sick animal has exposed other animals in the close proximity of the herd possibly spreading the disease to others.

As a result of the problems of early detection of disease, various telemetry sensing devices, often termed “telltale” devices, have been provided in the prior art. These devices are helpful for determining a diseased condition in an animal and may also be used for indicating estrus in certain domestic animals. Generally these devices measure a physiological condition such as temperature which is indicative of the health or other condition of the animal.

Some prior art systems were developed but found only limited acceptability in the cattle industry for various reasons. One reason that temperature sensing devices which are either ingested by the animal or inserted in a bodily canal have not been widely used is that they are either difficult to insert and may become easily dislodged or expelled by the animal. Implantable devices which are inserted in an incision in the animal also present problems of dislodgement. Further, because devices of this type require an open incision in which the monitoring device is placed, the incision may become easily infected, thus, contributing to a diseased condition in the animal.

Other prior systems use a transmitter and a receiver. The transmitter includes a physiological sensing device, such as a temperature sensor, and is contained in a small capsule-like housing with a suitable power supply and a transmitter. The capsule may be implanted in a suitable incision in the animal that heals over in a few days. The sensor monitors temperature and upon a predetermined limit or threshold value being reached, a signal indicative of the temperature of the animal is transmitted at low power and a low frequency. Other digitally coded information relating to the identity of the animal may also be transmitted by the implanted transmitter. However, these systems also offer the problems of possible infection due to the incision and the time and expense of inserting these transmitters into the animal.

Additionally, even though some devices were capable of monitoring certain health characteristics, these devices were not able to track animals, especially those in large numbers, such as dairy cows. There are approximately 11 million dairy cows in the United States. As a result, collars were developed that identify each animal and keeps track of it's movement. Approximately 50% of these animals are outfitted with a collar (costing from $30-50) that identifies the animal with a number and keeps track (through a pedometer type device) the amount of walking done by the animal. Again, due to the expense, these devices are not practical and these devices are also not capable of monitoring the health of the animal in a non-invasive manner.

Accordingly, what is needed is a novel animal monitoring device that is cost effective and capable of accurately monitoring the movement and/or health parameters of an animal. Also, what is needed is an animal monitoring device that may be used to monitor an animal in a safe and non-invasive manner.

### SUMMARY OF THE INVENTION

The invention provides an animal monitoring device that is cost effective and capable of accurately monitoring the movement and/or health parameters of an animal. the device may be easily and securely attached to an animal in a manner that does not adversely affect or harm the animal. Using microsensor or nanosensor technology, the device of the present invention is able to monitor various health indicators.

In particular, the present invention provides an animal monitoring device that is capable of performing one or more of the following: identifying the animal by number, keeping track of its movement, monitoring ketone emissions, monitoring vital functions such as body temperature, nose wetness, and ambient temperature and humidity. These measurements provide a unique indication of the health of the animal. Through data logging hardware, the present invention would be capable of logging these clinical health indicators for at least an eight hour period. In an additional embodiment, when the cow enters a stall at a milking facility, the milking computer and data acquisition system may download this information through wireless or hardwired technology. The data may be analyzed and used as an indicator of animal health.

More particularly, in one embodiment, the present invention provides an animal monitoring device including a housing, a ketone sensor contained within the housing for measuring ketone emissions, and a control system for monitoring the at least one sensor.

### DETAILED DESCRIPTION OF THE DRAWINGS

Other objects, features and advantages of the will become apparent upon reading the following detailed description, while referring to the attached drawings, in which:

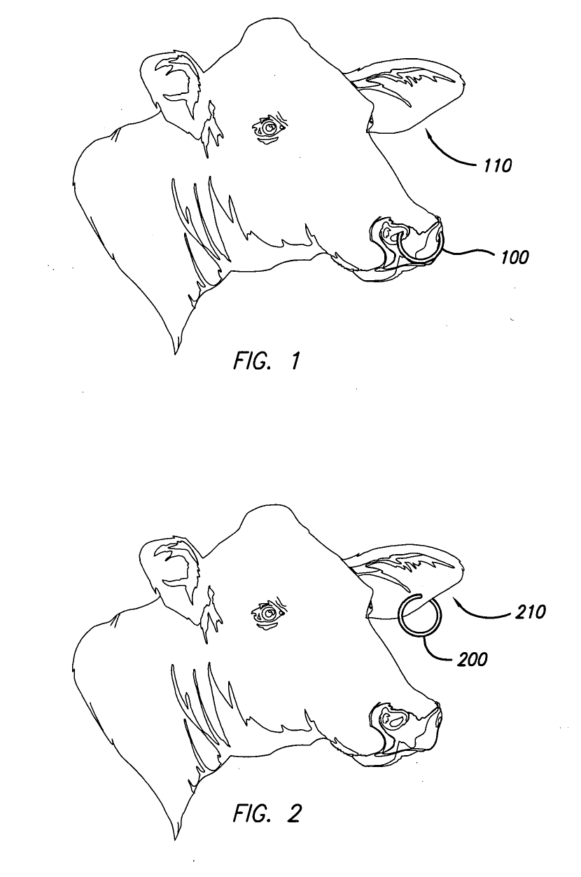

FIG. 1 is a view of one embodiment of an animal monitoring device as attached to an animal in accordance with one aspect of the present invention.

FIG. 2 is a view of another embodiment of an animal monitoring device as attached to an animal in accordance with another aspect of the present invention.

### DETAILED DESCRIPTION OF THE INVENTION

The present invention is more particularly described in the following description and examples that are intended to be illustrative only since numerous modifications and variations therein will be apparent to those skilled in the art. As used in the specification and in the claims, the singular form “a,” “an,” and “the” may include plural referents unless the context clearly dictates otherwise. Also, as used in the specification and in the claims, the term “comprising” may include the embodiments “consisting of” and “consisting essentially of.”

The present invention provides an animal monitoring device that may be outfitted in one embodiment as a nose ring. An advantage of nose ring is an ability to measure ketone emissions. Chronic inhalation studies of methyl ethyl ketone in animals have reported slight neurological, liver, kidney, and respiratory effects. As such, by using a nose ring, a ketone sensor may be utilized in the present invention. In alternative embodiment, the animal monitoring device that may be outfitted as an ear tag.

The devices of the present invention may be capable of not only identifying the animal by number and keeping track of its movement, but it may also capable of monitoring ketone emissions and/or vital health indicators of the animal. These health indicators may include, but are not limited to, body temperature, nose wetness, ambient temperature, humidity, and a combination thereof. By tracking these measurements, it is possible to provide a unique indication of the health of the animal. Through data logging hardware, this device would be able log these clinical health indicators for a sufficient period of time.

In an alternative embodiment, the present invention may be used in an animal processing facility to track the health of the animal. For example, if the animal that is being monitored is a dairy cow, the device of the present invention may be used such that when the cow enters a stall at a milking facility, the milking computer and data acquisition system may download this information through wireless or hardwired technology. The data may be analyzed and used as an indicator of animal health.

The device of the present invention includes sensors that are used measure various health characteristics. In one embodiment, these sensors utilize microsensor and/or nanosensor technology to monitor one or more of the health indicators listed above.

In addition to the sensors, the devices of the present invention also include a control system. The control system is programmed to take measurements from the ketone sensor, as well as any other sensors to monitor different health and/or ambient condition measurements from the animal at various times. In addition, the control system may be programmed to transmit this information on a periodic basis to track the animal and/or the health of the animal.

In an additional embodiment, the control system may be programmable such that it may be adapted to a variety of power systems, such as the wireless and hardwired systems previously mentioned. The system may be forward compatible permitting it to adjust to changes in technology.

The animal monitoring device of the present invention may also include means for identifying and/or numbering each animal. For example, a radio frequency identification (RFID) tag may be included in the device and may be programmed with information regarding the animal, the specific breed of animal, a unique identifier, and a combination thereof. Other means of tracking and/or identifying an animal may also be used in lieu of an RFID tag, such as a bar code system.

The present invention may also include a means for supplying power to the device. These means may include a battery or may comprise a self-regenerating power supply, such as a solar panel or the like that is used to power the system. Alternatively, the device may comprise a passive device such that the device is activated by a signal, such as a radio frequency, and a portion of the signal is converted to energy by the device. This converted energy may then be used to perform and monitoring and/or data transmission by the device.

The present invention also includes a housing. The housing is used to contain all of the components in the device, such as any sensors, identification means, control system, power supply, and combination thereof. The housing may be in the form of a ring or tag and may be applied to any selected location of the animal, such as the ear or nose. The housing may also provide protection to these components from the weather and/or contact with other animals. It is contemplated that in select embodiments, the housing is constructed from a durable material, such as a plastic material.

The present invention may be incorporated into a nose ring or an ear tag or any similar device that is capable of being attached to an exterior of the animal. Using a nose ring or an ear tag permit the animal to be tracked and/or monitored in a quick manner without performing an invasive procedure on the animal. As such, the present invention is safer to animals and, therefore, more humane. Additionally, as the ring or tag may be quickly and securely attached to the animal, the present invention provides costs and time savings over prior art monitoring systems. Lastly, using an external monitoring device permits quick replacement of the device and/or any power system within the device, such as a battery, should repair and/or replacement of the device become necessary.

Reference is now made with specific detail to the drawings in which like reference numerals designate like or equivalent elements throughout the several views, and initially to FIG. 1.

FIG. 1 is a perspective view of one embodiment of an animal monitoring device 100 in accordance with one aspect of the present invention. The device is attached to an animal 110, such as a cow, in a manner that enables the device to monitor selected health indicators of the animal. As shown in FIG. 1, the device 100 is attached to the cow's nose. As such, the device is able to measure ketone emissions for the animal. If additional sensors are included, these may be selected to measure various physiological and/or ambient conditions, such as body temperature, nose wetness, ambient temperature and/or humidity. In addition, the device may monitor other information regarding the animal, such as the animal number and movement.

FIG. 2 is a perspective view of one embodiment of an animal monitoring device 200 in accordance with one aspect of the present invention. The device is attached to an animal 210, such as a cow, in a manner that enables the device to monitor selected health indicators of the animal. As shown in FIG. 1, the device 200 is attached to the cow's ear. As such, the device may measure body temperature and ambient temperature and humidity, as well as animal number and movement.

The present invention may also be used in a system for controlling and operating a cow-milking carousel. The carousel may have an animal-identity confirmation device at the entrance and read the animal-monitoring device of the present invention. The carousel may have a number of milking stands wherein teat cups are applied to the animals at the milking stands and are automatically removed once the animals have been milked. As an animal enters a milking stall, the device may be read and the animal identified. In one embodiment, the animal would be milked only if the control system registers that the animal had not been milked recently. In an alternative embodiment, should the sensors read that the animal is not healthy, the system may cause the animal to be by-passed rather than milking a sick animal. In yet another alternative embodiment, a notification may be sent to an operator indicating the presence of the sick animal such that the animal may be removed from the herd and/or properly cared for rather risking the health of the rest of the herd.

The control system of the present invention has a multitude of other uses besides in conjunction with an animal monitoring device. The system may also be used to provide information to primary and auxiliary braking systems, fuel injection systems, steering and suspension systems and tire inflation systems.

Although the illustrative embodiments of the present disclosure have been described herein with reference to the accompanying drawings and examples, it is to be understood that the disclosure is not limited to those precise embodiments, and various other changes and modifications may be affected therein by one skilled in the art without departing from the scope of spirit of the disclosure. All such changes and modifications are intended to be included within the scope of the disclosure as defined by the appended claims.

1. An animal monitoring device comprising:
a housing;
a ketone sensor contained within the housing for measuring ketone emissions; and
a control system for monitoring the at least one sensor.

2. The animal monitoring device of claim 1, further comprising an identification system for identifying the animal.

3. The animal monitoring device of claim 2, wherein the identification system is also capable of tracking the animal.

4. The animal monitoring device of claim 2, wherein the identification system is selected from a radio frequency identification tag.

5. The animal monitoring device of claim 2, wherein the control system is also capable of transmitting information from the ketone sensor and the identification system.

6. The animal monitoring device of claim 1, further comprising a power source.

7. The animal monitoring device of claim 6, wherein the power source is selected from a battery, a solar panel, or a combination thereof.

8. The animal monitoring device of claim 7, wherein the power source includes a solar panel.

9. The animal monitoring device of claim 1, further comprising at least one additional sensor.

10. The animal monitoring device of claim 9, wherein the at least one additional sensor is selected from a body temperature sensor, a nose wetness sensor, an ambient temperature sensor, a humidity sensor, or a combination thereof.

11. The animal monitoring device of claim 9, wherein the control system is also capable of transmitting information from the at least one additional sensor.

12. The animal monitoring device of claim 1, wherein the housing is in a shape of a nose ring that is capable of being secured to a nose of an animal such that ketone emissions may be monitored by the ketone sensor.
